(19) United States
(12) Patent Application Publication
(10) Pub. No.: US 2011/0281298 A1
(43) Pub. Date: Nov. 17, 2011

(54) METHOD AND APPARATUS TO EXTRACTED AND REDUCE DISSOLVED HEMI-CELLULOSIC SOLIDS IN BIOMASS FOLLOWING PRE-HYDROLYSIS

(75) Inventors: Joseph Rawls, Atlanta, GA (US); Thomas Pschorn, Lennoxville (CA); Michael Kingsley, Corinth, NY (US)

(73) Assignee: ANDRITZ INC., Glens Falls, NY (US)

(21) Appl. No.: 13/103,341

(22) Filed: May 9, 2011

Related U.S. Application Data
(60) Provisional application No. 61/333,481, filed on May 11, 2010.

(51) Int. Cl. C12P 1/00; B08B 3/14; B01J 8/08

(52) U.S. Cl. 435/41; 422/232; 134/13

(57) ABSTRACT

A method to wash and remove dissolved solids from biomass including: discharging a biomass slurry from a pretreatment vessel to a biomass slurry retention device; adding recovered wash liquid to dilute the biomass slurry in the retention device, wherein the recovered wash liquid is extracted from a drainer device upstream of the first retention device; discharging the diluted biomass slurry from the retention device to the drainer device; separating wash liquid with dissolved solids from the diluted biomass slurry in the drainer device and discharging a concentrated biomass slurry from the drainer device, and recovering the wash liquid from the diluted biomass slurry in the drainer device and transferring the recovered wash liquid to the retention device.

### CROSS RELATED APPLICATION

This application claims the benefit of application Ser. No. 61/333,481 filed May 11, 2010, which is incorporated in its entirety by reference.

### BACKGROUND OF THE INVENTION

The present invention relates to pre-hydrolysis of cellulosic biomass to extract five carbon sugars (C5) and six carbon sugars (C6) for production of bio-fuels and chemicals. In particular, the invention relates to methods and devices for early extraction of hemi-cellulosic sugars dissolved from the biomass during pre-hydrolysis and washing the dissolved solids from the biomass discharged from a pre-hydrolysis vessel.

Biomass includes lignocellulosic material such as wood, including wood chips and sawdust, and fibrous plants. Biomass feed stock is the biomass material conveyed to an inlet of a processing vessel, such as a pre-hydrolysis vessel. Biomass also includes agricultural residues (such as stalks, stover and hulls), straws and grasses or forest and sawmill residues (wood chips and shredded thinnings). Biomass typically excludes fossil fuels which have been transformed by geological processes into substances such as coal or petroleum. Biomass can be grown from numerous types of plants, including miscanthus, switch grass, hemp, corn, poplar, willow, sorghum, sugarcane, and varieties of tree species, ranging from eucalyptus to oil palm (palm oil).

Pre-hydrolysis, which may be performed as auto-hydrolysis, refers to cooking of cellulosic biomass feed stocks at elevated temperatures of, for example, 110 degrees Celsius (° C.) to 160° C., for approximately 10 to 120 minutes in a solution that dissolves and hydrolysizes hemi-cellulose from the biomass. Auto-hydrolysis refers to using acetic acid released from the acetyl groups in biomass during auto-hydrolysis conditions.

To enhance the pre-hydrolysis of hemi-cellulose mild acids, such as SO₂-gas, oxygen and compressed air along with ammonia or other catalyzing agents may be added to the pre-hydrolysis reactor vessel. The pre-hydrolysis and auto-hydrolysis reactions dissolve and separate hemi-cellulose in the biomass to form dissolved C5 sugars, such as xylose and arabinose, and amorphous sugars. Pre-hydrolysis and auto-hydrolysis of soft-wood hemi-cellulose typically yields gluco-mannan, and of hard woods yields xylose and arabinose.

A conventional pretreatment vessel is a pre-hydrolysis reactor vessel that receives cellulosic biomass feed stock, subjects the biomass feed stock to a hydrolysis reaction, and discharges a slurry of liquid and biomass to an optional bin or other intermediate storage silo or tank, or to a life bottom hopper or similar (with the optional provision to pre-steam) with a discharge device and feeder. The intermediate storage devices may operate under elevated pressures or temperatures of, for example, 110 degrees Celsius (° C.) to 160° C.

From these intermediate storage devices or directly from the pretreatment vessel, prehydrolysed biomass may be fed to a draining device, such as an inclined drainer, such as a pressing device, which feeds the slurry of biomass and liquid to a sealing device. Conventionally, the draining device removes liquid from the biomass (“dewatering”) to increase the suspended solids (SS) level in the biomass, and does not change the dissolved solids (DS) level of the biomass. The sealing device may be a rotary valve, a modular screw device (MSD—such as the Impressafiner™ sold by the Andritz Group), another high compression, extruder like screw device, or a plug screw feeder that feeds the biomass under pressure to a subsequent reactor vessel, such as a fermentation vessel, a hydrolysis reactor or cooking vessel, e.g., digester.

Some liquor may be conventionally extracted from the pretreatment vessel to extract a portion of the dissolved hemi cellulosic material from biomass. The biomass discharged from the pretreatment vessel is conventionally transferred directly to a sealing device, e.g., a MSD, and then to subsequent processing, such as to enzymatic hydrolysis or fermentation stages or to a second stage pretreatment stage—for example a high pressure reactor—which may discharge the cooked biomass via steam-explosion or just be diluting and discharging as a slurry.

The fermentation stages are intended to generate alcohols, e.g. ethanol, from cellulosic feed stocks, e.g., biomass, where high concentrations of a catalyst, e.g., acids, are used and which stages occur at high temperatures and pressures. Removing inhibitors to fermentation, like aldehydes (such as HMF, furfural, and formaldehyde), monomeric phenolics (such as vanillin and coniferylaldehyde), hemi-cellulosic compounds, acids (such as acetic acid, and formic acid) and other components and chemicals should increase the alcohol yield in the following fermentation.

### BRIEF DESCRIPTION OF THE INVENTION

Hemi cellulose is dissolved from biomass feed stock in a pre-hydrolysis reactor or auto-hydrolysis reactor. After the pre-hydrolysis or auto-hydrolysis reactor, the biomass is washed and dewatered (and optionally washed and dewatered repeatedly) to extract the dissolved hemi-cellulose (C5 sugars) and thereby separate the C5 sugars in the biomass from other sugars, e.g., C6 (glucose), in the biomass. The washing and dewatering of the biomass, and the associated extraction of the hemi cellulose, is performed before the biomass is transferred to a sealing device, e.g., MSD, and further process stages, such as fermentation.

Extracting hemi-cellulose facilitates the subsequent conversion of other separated sugars to other products. For example, the C5 and C6 sugars separated from hardwoods, herbaceous biomass and agricultural residues may be converted to xylose and other food additives, biogas (through aerobic or anaerobic fermentation), methyl-furan (for use as a high octane oxygenate) or as aqueous sugars for conversion with micro-organisms to alcohols, e.g. ethanol.

If dissolved hemi-cellulose produced in a pre-hydrolysis reactor is not extracted before the biomass is further processed in fermentation or other processes, the fermentation or other processes may convert the C5 sugars to components and chemicals that inhibit subsequent fermentation steps, such as the fermentation of the extracted C6 sugars. This inhibition of fermentation occurs in conventional processes that include pre-hydrolysis stages and pre-treatment processes in which the C5 sugars are not extracted before the fermentation stages.

A method is disclosed herein to wash and remove dissolved solids from biomass comprising: discharging a biomass slurry from a pretreatment vessel to a biomass slurry retention device; adding recovered wash liquid to dilute the biomass slurry in the retention device, wherein the recovered wash liquid is extracted from a drainer device upstream of the first retention device; discharging the diluted biomass slurry from the retention device to the drainer device; separating wash liquid with dissolved solids from the diluted biomass slurry in the drainer device and discharging a concentrated biomass slurry from the drainer device, and recovering the wash liquid from the diluted biomass slurry in the drainer device and transferring the recovered wash liquid to the retention device.

In the method, the pretreatment vessel may be a pre-hydrolysis reactor in which the biomass slurry undergoes hydrolysis, and the recovered wash liquid includes hemi cellulosic material dissolved from the biomass slurry. The method may include discharging a portion of the recovered wash liquid to a hydrolysate recovery device. In the method, the retention device may be a pressurized tank or a pressurized dilution conveyor and the drainer device is an inclined drainer including a screw or auger or an inclined pressing device, a lower inlet for the biomass, an upper outlet for the biomass and a lower liquid outlet. In the method, a portion of the recovered wash liquid is introduced to a lower portion of the pre-hydrolysis reactor vessel or other pretreatment vessel.

The method may further comprise: introducing the concentrated biomass slurry from the drainer to a second retention device; adding wash liquid recovered from a second drainer upstream of the second retention device to dilute the concentrated biomass slurry in the second retention device; discharging the diluted biomass slurry from the second retention device to the second drainer device; separating wash liquid with dissolved solids from the diluted biomass slurry in the second drainer device and discharging a concentrated biomass slurry from the second drainer device, and recovering the wash liquid from the diluted biomass slurry in the second drainer device and transferring the recovered wash liquid to the second retention device.

The method may further comprise the introduction of a catalyst or a solvent before or between the various washing stages. The retention device and the inclined draining device may maintain the biomass at elevated temperatures and pressures to improve the diffusion of dissolved solids in the biomass, to enhance washing of the biomass and for energy efficiency by reducing the need to add heat or steam in a subsequent processing vessel.

The method may further comprise: discharging the concentrated biomass slurry from one of the drainer device or the second drainer device to a high compression extruder; compressing the concentrated biomass slurry in the high compression extruder to further concentrate the biomass slurry, and recovering the wash liquid from the high compression extruder. In the method, the concentrated biomass slurry or the further concentrated biomass slurry may be transferred to a liquefaction reactor where the cellulose will be converted into C6 sugars (hydrolyzed through enzymes or organisms) or a fermentation unit in which C6 sugars in the biomass ferment.

A system is disclosed for washing processed biomass and removing dissolved solids from the biomass comprising: a pre-hydrolysis reactor vessel having a lower outlet to discharge a biomass slurry, wherein the pre-hydrolysis reactor vessel is operated at conditions that promote hydrolysis of the biomass; a retention tank coupled to a conduit to receive the biomass slurry discharged through the lower outlet of the pre-hydrolysis reactor vessel, wherein the retention tank receives recovered wash liquid and adapted to dilute the biomass slurry in the tank with the recovered wash liquid and is adapted to discharge diluted biomass slurry; a drainer having a lower inlet coupled to a conduit adapted to transfer the discharged diluted biomass slurry from the retention tank to the inlet to the drainer, an upper solids outlet to discharge concentrated biomass slurry, and a lower liquids drain to discharge the recovered wash liquid, wherein the lower liquids drain is coupled to a conduit to transfer the recovered wash liquid to the retention tank.

The system may further comprise a hydrolysate recovery tank coupled to the conduit for the recovered wash liquid. The retention device may be a pressurized tank and the drainer device is an inclined drainer including a screw or auger, between the lower inlet and the upper solids outlet. The pre-hydrolysis reactor vessel may include a nozzle on a lower portion of the reactor vessel and coupled to the conduit to receive the recovered wash liquid. The system may further comprise: a high compression extruder having an inlet coupled to a conduit for the discharged concentrated biomass slurry from the drainer.

### BRIEF DESCRIPTION OF THE DRAWINGS

FIGS. 1A and 1B are a flow chart of an exemplary process for extracting hemi cellulosic components from biomass and reducing the dissolved solids content of biomass following pre-hydrolysis.

### DETAILED DESCRIPTION OF THE INVENTION

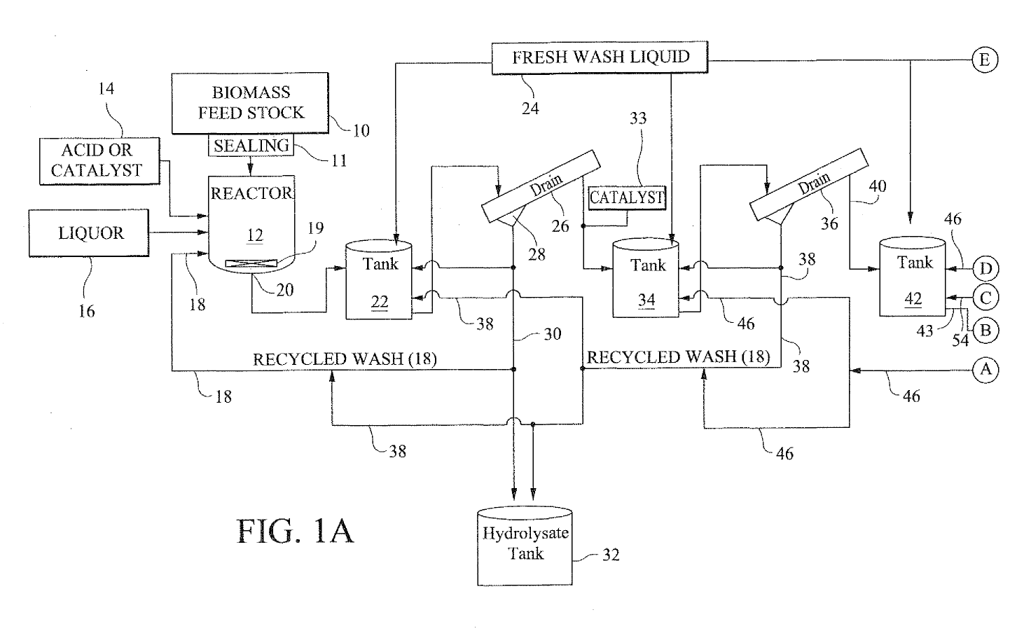

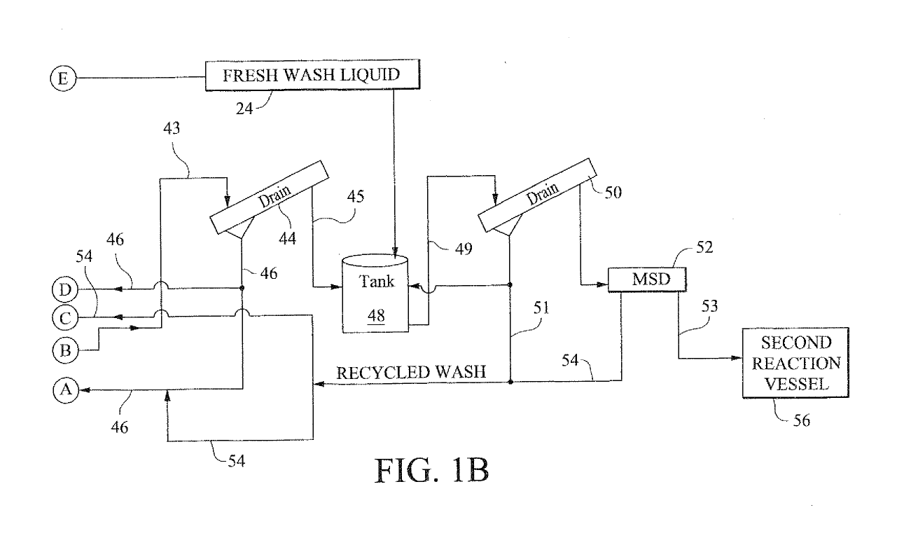

FIGS. 1A and 1B are a flow chart of an exemplary process for extracting hemi cellulosic components from biomass and reducing the dissolved solids content of biomass following pre-hydrolysis.

A biomass supply 10, e.g., a chip bin, provides biomass feed stock to a pretreatment vessel 12, such as a pre-hydrolysis or auto-hydrolysis reactor vessel. The biomass feedstock 10 may be conveyed to the pressurized vessel 12 by gravity or mechanically, e.g., via a screw conveyor or a conveyor belt.

The biomass feed stock may be conveyed to the pretreatment vessel 12 as dry biomass material or with a partial liquid content. The feeding of the biomass feed stock may include an optional injection of pre-steam and a sealing device that pressurizes the biomass to a pressure suitable for feeding to the vessel 12. The sealing device 11 may be a rotary valve, a MSD Impressafiner™ (which is a high compression, extruder like screw device) or a plug screw feeder. Alternatively, the biomass feed stock may be pumped directly to a downstream reactor vessel, e.g., a fermentation vessel in a manner similar to a turbo-feed system used in pulping processes.

The pre-hydrolysis vessel 12 may be a horizontal, vertical or inclined reactor. A horizontal or inclined reactor may include an internal auger or screw to move the biomass slurry through the reactor. A vertical reactor may be similar in structure to a continuous digester vessel conventionally used for producing pulp from wood chips.

The pretreatment reactor vessel 12 may be a sealed vessel operating at an elevated temperature of, for example, above 100 degrees Celsius (100° C.) and under pressure above atmospheric pressure. The reaction conditions inside the pretreatment reactor vessel 12, e.g., a pre-hydrolysis reactor, may include a temperature in a range of 110 degrees Celsius (° C.) to 160° C. and a pressure in a range of 1.5 gauge bar to 6 gauge bar. The retention period of biomass in the vessel 12 may be in a range of 10 minutes (min) to 120 min. The retention time is the period from when the biomass enters the pretreatment reactor vessel 12 to when the biomass is discharged from the vessel. These reaction conditions and retention times are exemplary. The reaction conditions in an operational embodiment of the pre-treatment reactor vessel will depend on the biomass material being processed and conditions specific to that process.

Mild acids, SO₂-gas, oxygen, compressed air, ammonia or other catalyzing agents 14 may be optionally added to the pretreatment reactor vessel to promote the hydrolysis reaction of the biomass in the vessel. Alternatively, the biomass may undergo an auto-hydrolysis using, for example, the acetic acids released from the acetyl groups in the biomass under the auto-hydrolysis conditions.

Combinations of steam, ammonia and other heating medium(s) may be used to provide heat energy to the pressurized reactor vessel 12 or to the biomass feed stock 10 prior to entering the vessel 12. The addition of heat may be unnecessary if the biomass feedstock 10, liquor 16 and pressure conditions in the pretreatment vessel 12 are sufficient to elevate the temperature in the vessel to promote hydrolysis.

Various liquors 16, e.g. chemicals, water and other liquids, may be added to the biomass in the treatment vessel. For example, acids, e.g., sulfur-dioxide SO₂, may be added to promote a hydrolysis reaction in the vessel. The liquid, chemicals and biomass form a biomass slurry that is discharged from the pretreatment reactor vessel at the vessel bottom outlet 14.

The flow rates of solids and liquor through the pretreatment reactor vessel depend on the individual conditions of the process for treating the biomass and can be determined by a person of ordinary skill in the art of treating biomass. The proportion of suspended solids (SS) in the pretreatment vessel may be 20% solids and 80% liquids. The liquid in the slurry may, for example, contain twenty five percent (25%) dissolved solids (DS). The dissolved solids in the slurry include the hemi cellulosic material extracted from the fibers in the biomass during the hydrolysis process occurring in the pretreatment vessel.

The process shown in FIGS. 1A and 1B are envisioned to provide a continuous flow of biomass through the pretreatment vessel and the subsequent tanks and drainers. Alternatively, the process disclosed herein may be embodied as a batch process in which biomass sequentially and in separate steps fills, is treated and thereafter is discharged from the pretreatment reactor vessel. In a batch process, the biomass discharged from the pretreatment vessel cyclically and periodically flows to the tanks and drainers downstream of the pretreatment vessel.

Recovered wash liquid 18 is introduced at or near the bottom of the pretreatment reactor vessel 12. The recovered wash liquid may have a lower dissolved solids content, e.g. about 18%, than the biomass slurry in the upper portion of the pretreatment vessel. The addition of recovered wash liquid reduces the portions of solids in the biomass slurry at the bottom outlet 20 of the vessel 12 to, for example, 12% suspended solids and 20% dissolved solids. The recovered wash liquid 18 may also cool the biomass slurry to suppress the hydrolysis reaction in the biomass as the biomass is discharged from the vessel.

A discharge scraper or a discharge screw 19 in a lower region of the interior of the pretreatment reactor vessel assists in discharging the slurry of biomass from bottom discharge outlet 20 of the pretreatment reactor vessel 12.

The biomass slurry discharged from the pretreatment reactor vessel is washed to remove chemicals and dissolved solids and to extract hydrolysate, e.g., hemi-cellulosic (C5 sugars). From the bottom outlet 20 of the pretreatment reactor vessel 12, the biomass slurry flows under the force of gravity to an upper inlet of a first retention tank 22. The retention tanks, e.g., 22, may be pressurized to a pressure the same as or similar to the pressure in the pretreatment reactor vessel 12 to maintain pressure on the biomass material and retain heat energy in the biomass material. The temperature in the retention tanks 22, 34, 42, 48 may be above 100° C. The biomass slurry flowing to the tank 22 may have a suspended solids content of 12% and a dissolved solids content of 20%.

The first retention tank 22 receives recovered wash liquid 18 from biomass drainers 26, 36, 44 and 50 that are downstream of the tank 22. The drainers are devices that extract liquid from the biomass, and may include screws, augers, presses and other devices that pull liquid out of the biomass.

The wash liquid from the drainers is recovered by being used in the tanks upstream in the process of where the wash liquid is extracted from the biomass. The recovered wash liquid has a lower or equal dissolved solids (DS) level as compared to the dissolved solids level of the biomass in the first retention tank. For example, the recovered wash liquid 18 added to the first retention tank 22 may have a DS level of 18% as compared to the DS level of 20% of the biomass entering the upstream retention tank(s). Fresh wash liquid 24, such as water, may be added to each of the retention tanks 22, 34, 42 and 48.

The combination of fresh wash liquid and recovered wash liquid dilutes the slurry of biomass in the retention tanks, and promotes the migration of hemi cellulose and other solids from the fibers in the biomass to the liquid in the biomass slurry. The biomass may be retained for a period of, for example, 3 to 15 minutes, in the first retention tank 22.

The biomass settles at the bottom of the first retention tank 22 and is discharged from the bottom to a first drainer 26, such as MSD screw press. The dissolved solids content of the biomass slurry discharged from the first retention tank is lower than the biomass slurry entering the tank. For example, the biomass slurry at the discharge of the tank 22 may have a DS level of 18% as compared to a DS level of 20% at the inlet to the tank. The suspended solids (SS) level of the biomass slurry also drops in the tank due to the addition of fresh and wash liquids to the tank. For example, the SS level of the biomass slurry at the discharge of the tank 22 may be 8% as compared to a SS level of 12% at the inlet to the tank.

The drainer 26 dewaters the biomass material to reduce its suspended solids (SS) level. For example, the drainer may reduce the SS level to 20% from 8% of the biomass slurry. If the drainer does not add wash liquid, the drainer does not change the dissolved solids (DS) level of the biomass.

If one or both of fresh and recovered wash liquid are added to the drainer, the DS level of the biomass may be reduced by the drainer. Wash liquid may be added to the drainer by nozzles mounted on the housing of the drainer to inject the wash liquid into the biomass move upwardly through the drainer. If the biomass in the drainer has sufficient retention time to allow solids, e.g., hemi cellulose, in the biomass to migrate to dissolved solids in the liquid, one or more of the retention tanks may be unnecessary.

The drainer 26 may include an internal screw or auger to move biomass material upwardly through an internal passage in the drainer. The screw or auger moves the biomass material upwardly while allowing liquids to drain to the bottom outlet 28. Further, the screen or auger may compress the biomass and thereby squeeze liquids with dissolved solids from the biomass. As the biomass moves up through the drainer, liquid and dissolved solids in the biomass remain in the bottom of the drainer and are extracted through the outlet 28. A screen between the internal passage in the drainer and the outlet prevents biomass solid materials, e.g., fibers, from flowing through the drain outlet.

The extracted wash liquid with dissolved solids, e.g., DS level of 18%, from the first drainer 26 flows through the outlet to a conduit 30 that directs a portion of the wash liquid to a hydrolysate tank 32 from which the dissolve hemi cellulosic (C5 sugars) material may be extracted and used in further processes. Another portion of the extracted wash liquid from the drainer is used as recovered wash liquid and directed to the pretreatment vessel 12 and to the first retention tank 22, both of which are upstream of the first drainer 26. The recovered wash liquid flows upstream, e.g., cross-current, to the flow direction of the biomass slurry through the series of retention tanks and drainers.

The sequence of diluting the biomass material with wash liquid to reduce the dissolved solids (DS) level and thereafter dewatering the biomass material to reduce the suspended solids (SS) level may be repeated until the DS and SS levels of the biomass material is at desired levels.

A second retention tank 34 receives the dewatered biomass slurry from the first drainer 26 and dilutes the biomass with fresh wash liquid 24 and wash liquid recovered from drainers downstream of the second retention tank. The structure, operation and purpose of the second retention tank is substantially the same as the first retention tank, with the exception that the second retention tank receives the biomass slurry from a drainer and at a higher SS level and a lower DS level than the biomass slurry received by the first tank 22 from the pretreatment vessel 12. For example, the biomass slurry flowing to an upper inlet of the second retention tank 34 may have a SS level of 20% and a DS level of 18%, as compared to the biomass slurry with an SS level of 12% and a DS level of 20% entering the first retention tank.

The DS level in the biomass slurry is reduced by sequentially (1) diluting the biomass slurry to reduce the DS level and (2) dewatering the biomass slurry by extracting wash liquid with dissolved solids. These two steps reduce the DS content of the biomass slurry, extract dissolved hemi cellulosic compounds and maintain the SS content to acceptable levels. The sequence of diluting and dewatering the biomass may be repeated with successive retention tanks and drainers until the DS level of the biomass has been reduced to a desired level, e.g., to below 10% and preferably below 8% and even below 7%. Alternatively, the sequence of diluting and dewatering the biomass slurry may be repeated until sufficient hemi cellulosic material is removed from the biomass.

The second retention tank 34 receives the dewatered biomass material from the first drain 26. Fresh wash liquid 24 and recycled wash liquid from a second drainer 36 and subsequent drainers are added to the second retention tank 34 to dilute the biomass slurry in the tank. The biomass slurry fed to an upper inlet to the second retention tank may have a DS level of 18% and a SS level of 20%. The biomass slurry may be discharged from the second retention tank with a DS level of 14% and a SS of 5%. The second drainer 36 dewaters the biomass slurry such that at the drainer discharge the slurry as a DS level of 14% and a SS level of 20%. The wash liquid outlet to the second drain 36 discharges liquid with dissolved solids, e.g., hemi cellulosic material, to a conduit 38 that feeds the wash liquid to one or more of the hydrolysate tank 32, the upstream retention tanks 22, 34, and the pretreatment reactor vessel 12.

The biomass discharged from an upper outlet of the second drainer 36 flows through conduit 40 to a third retention tank 42. The biomass slurry may enter the third retention tank 42 with a DS level of 14% and a SS level of 20%. Fresh wash liquid 24 and recovered wash liquid 46, 54 flows into the third retention tank 42 to dilute the biomass slurry. The retention time of the biomass slurry in the third retention tank 42, as well as all of the retention tanks 22, 34, 48, is sufficient to allow hemi cellulosic material and other solids in the fibers of the biomass to migrate into the liquid as dissolved solids. At the lower discharge of the third retention tank 42, the biomass slurry may have a DS level of 11% and a SS level of 5%. From the lower discharge the biomass slurry flows through conduit 43 to the third drainer 44.

The biomass slurry discharged from the third retention tank is dewatered in the third drainer 44, which may be an horizontal, inclined or vertical drainer with screw or auger conveyors (as may be each of the drainers). The drainers 26, 36, 44, 50 may also be embodied as a pressing device that compresses the discharged biomass slurry to extract water (or other liquid) from the slurry.

The third drainer 44 has an upper outlet that discharges biomass slurry having, for example, a DS level of 11% and a SS level of 20%. The third drainer has a lower liquid outlet that discharges extracted wash liquid to a conduit 46. The discharged wash liquid may have a DS level of 11%, which is the same DS level as the biomass slurry moving through the drainers. Each of the drainers 26, 36 44 and 50, generally discharge wash liquid having a DS level the same as the DS level of the biomass slurry passing through the drainer. The wash liquid discharged from the third drainer flows through conduit 46 to the hydrolysate tank 32 and the upstream retention tanks 34, 42 and pretreatment reactor vessel 12.

A fourth retention tank 48 receives biomass slurry discharged to conduit 45 from the third inclined drainer 44. Fresh wash liquid 24 and recovered wash liquid from a discharge conduit 51 from a fourth drainer 50 is added to the fourth retention tank 48 to dilute the biomass in that tank. The biomass slurry entering the fourth retention tank 48 may have a DS level of 11% and a SS level of 20%. The biomass slurry discharged from the fourth retention tank may have a DS level of 7.4% and a SS level of 5%.

The fourth drainer 50 receives and dewaters the biomass discharged to conduit 49 from the fourth retention tank 48. The biomass discharged from an upper inlet to the fourth drainer may have a DS level of 7.4% and a SS level of 20%. The drainers may each elevate the SS level to a uniform level, such as to a SS level of 20%. The wash liquid extracted from the drainers has a DS level the same as the DS level of the biomass slurry flowing through the drainer.

The biomass slurry discharged from the last drainer, e.g., the fourth drainer 50, may flow via conduit 53 to a modular screw device (MSD) 52 or other high compression, extruder like screw device. An example of a MSD is the Impressafiner™ sold by the Andritz Group. The MSD 52 increases the SS level of the biomass slurry from, for example, 20% to 40%. The WASH liquid extracted from the biomass slurry by the MSD may flow through conduit 54 as recovered wash liquid that flows to the retention tanks and pretreatment reactor vessel 12. The recovered wash liquid form the MSD 52 may also flow to the hydrolysate tank 32.

The biomass slurry discharged from the MSD has a high SS level, e.g., 40%, a low DS level, e.g., 7.4%, and has had extracted much of its dissolved hemi cellulosic material. The biomass slurry from the MSD may flow to a second reaction vessel 56 in which further processes are performed on the biomass such as a fermentation in a fermentation vessel, or enzymatic hydrolysis in a hydrolysis reactor or further cooking in a cooking vessel, e.g., a digester vessel.

Four washing stages are shown in FIGS. 1A and 1B by way of example. The number of washing stages may be less than or greater than four, in other embodiments of the invention. The retention tanks, 22, 34, 42 and 48 may be pressurized tanks or other retention devices such as dilution conveyor, which is a pressurized vessel with an internal screw auger having cut-flights that evenly re-dilute the slurry inside the vessel of the conveyor. Further, a catalyst or a solvent may be added to the biomass slurry before or between the various washing stages, such as by introduction to the conduit between the screen of a drainer and a retention tank.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method to wash and remove dissolved solids from biomass comprising:
discharging a biomass slurry from a pretreatment reactor vessel to a biomass slurry retention device;
adding recovered wash liquid to dilute the biomass slurry in the retention device, wherein the recovered wash liquid is extracted from a biomass slurry drainer device upstream of the retention device;
discharging the diluted biomass slurry from the retention device to the drainer device;
separating wash liquid with dissolved solids from the diluted biomass slurry in the biomass slurry drainer device and discharging a concentrated biomass slurry from the biomass slurry drainer device, and
recovering the wash liquid from the diluted biomass slurry in the biomass slurry drainer device and transferring the recovered wash liquid to the retention device.

2. The method of claim 1 further wherein the pretreatment vessel is a pre-hydrolysis reactor in which the biomass slurry undergoes hydrolysis, and the recovered wash liquid includes hemi cellulosic material dissolved from the biomass slurry.

3. The method of claim 2 further comprising discharging a portion of the recovered wash liquid to a hydrolysate recovery device.

4. The method of claim 1 wherein the retention device is a pressurized tank and the drainer device is an inclined drainer including at least one of a screw and auger, a lower inlet for the biomass slurry, an upper outlet for the biomass slurry and a lower liquid outlet.

5. The method of claim 1 wherein the pretreatment vessel is a pre-hydrolysis reactor vessel and a portion of the recovered wash liquid is introduced to a lower portion of the pre-hydrolysis reactor vessel.

6. The method of claim 1 further comprising:
introducing the concentrated biomass slurry from the biomass slurry drainer device to a second biomass slurry retention device;
adding wash liquid recovered from a second biomass slurry drainer upstream of the second biomass slurry retention device to dilute the concentrated biomass slurry in the second biomass slurry retention device;
discharging the diluted biomass slurry from the second biomass slurry retention device to the second biomass slurry drainer device;
separating wash liquid with dissolved solids from the diluted biomass slurry in the second biomass slurry drainer device and discharging a concentrated biomass slurry from the second biomass slurry drainer device, and
recovering the wash liquid from the diluted biomass slurry in the second drainer device and transferring the recovered wash liquid to the second retention device.

7. The method of claim 1 further comprising:
discharging the concentrated biomass slurry from one of the drainer device or the second drainer device to a high compression extruder;
compressing the concentrated biomass slurry in the high compression extruder to concentrate the biomass slurry, and
recovering the wash liquid from the high compression extruder.

8. The method of claim 1 further comprising introducing a catalyst or a solvent to the biomass slurry prior to introducing the biomass slurry to the retention device or the second retention device.

9. The method of claim 1 further comprising adding heat energy to or maintaining an elevated pressure on the biomass slurry while the biomass slurry is in the retention device or in the drainer device.

10. The method of claim 1 further comprising transferring the concentrated biomass slurry or the further concentrated biomass slurry to a fermentation unit in which C6 sugars in the biomass ferment.

11. A system for washing processed biomass and removing dissolved solids from the biomass comprising:
a pre-hydrolysis reactor vessel having an outlet to discharge a biomass slurry, wherein the pre-hydrolysis reactor vessel is operated at conditions that promote hydrolysis of the biomass;
a retention tank receiving the biomass slurry discharged through the outlet of the pre-hydrolysis reactor vessel, wherein the retention tank receives recovered wash liquid and is adapted to dilute the biomass slurry in the tank with the recovered wash liquid and discharge diluted biomass slurry;
a drainer including a biomass slurry inlet receiving the discharged diluted biomass slurry from the retention tank, a solids outlet to discharge concentrated biomass slurry, and a liquid drain to discharge liquid extracted from the discharged diluted biomass slurry, wherein the liquid drain is in fluid communication with the retention tank such that the discharged liquid flows to the retention tank.

12. The system of claim 11 further comprising a hydrolysate recovery tank in fluid communication with the liquid drain.

13. The system of claim 11 wherein the retention device is a pressurized tank and the drainer device is an inclined solids passage including a rotating screw or auger, wherein the drainer device includes a lower inlet coupled to a conduit to the outlet of the retention tank, an upper solids outlet coupled, and a screen separating the solids passage and the liquid drain.

14. The system of claim 11 wherein the pre-hydrolysis reactor vessel includes a nozzle on a lower portion of the reactor vessel which receives the discharged liquid.

15. The system of claim 11 further comprising a high compression extruder having an inlet coupled to receive the discharged concentrated biomass slurry from the drainer.

16. A system to process biomass comprising:
a pre-hydrolysis reactor vessel having an outlet to discharge a biomass slurry, wherein the pre-hydrolysis reactor vessel is operated at conditions that promote hydrolysis of the biomass;
a biomass slurry retention tank coupled to a biomass slurry conduit which is coupled to the outlet of the pre-hydrolysis reactor vessel and to a recovered liquid conduit and said tank includes a discharge outlet, wherein the retention tank receives recovered liquid and the biomass slurry from the reactor vessel, and the retention tank is adapted to dilute the biomass slurry with the recovered liquid and discharge diluted biomass slurry from the discharge outlet;
a drainer including a biomass slurry passage, a biomass slurry inlet at an end of the passage to receive the discharged diluted biomass slurry from the retention tank, a biomass slurry outlet at an opposite end of the passage to discharge concentrated biomass slurry, and a liquid drain to discharge recovered liquid extracted from the discharged diluted biomass slurry as the slurry moves through the passage, wherein the liquid drain is separated from the passage by a screen;
a recovered liquid conduit coupled to the liquid drain in fluid communication with the retention tank such that the recovered liquid flows to the retention tank, and
a hydrolysate recovery tank coupled to the recovered liquid conduit to receive the recovered liquid from the drainer.

17. The system of claim 16 wherein the retention device is a pressurized tank and the drainer is an inclined drainer having including a rotating screw or auger within the passage.

18. The system of claim 16 wherein the pre-hydrolysis reactor vessel includes a nozzle on a lower portion of the reactor vessel which receives the recovered liquid.

19. The system of claim 16 further comprising a high compression extruder having an inlet coupled to receive the discharged concentrated biomass slurry from the drainer.

20. The system of claim 16 further comprising:
a second retention tank coupled to a conduit extending to the biomass slurry outlet of the drainer to receive the concentrated biomass slurry from the drainer, wherein the second retention tank receives recovered liquid from a second drainer and discharges diluted biomass slurry;
the second drainer receiving the discharged diluted biomass slurry from the second retention tank and discharging concentrated biomass slurry, wherein the second drainer includes a liquid outlet to discharge liquid extracted from the biomass slurry in the second drainer,
wherein liquid extracted from the second drainer flows into both of the retention tanks.
